(19) United States
(12) Patent Application Publication
(10) Pub. No.: US 2024/0296552 A1
(43) Pub. Date: Sep. 5, 2024

(54) SYSTEMS AND METHODS FOR CARDIAC MOTION TRACKING AND ANALYSIS

(71) Applicant: Shanghai United Imaging Intelligence Co., Ltd., Shanghai (CN)

(72) Inventors: Xiao Chen, Lexington, MA (US); Shanhui Sun, Lexington, MA (US); Zhang Chen, Brookline, MA (US); Yikang Liu, Cambridge, MA (US); Arun Innanje, Lexington, MA (US); Terrence Chen, Lexington, MA (US)

(73) Assignee: Shanghai United Imaging Intelligence Co., Ltd., Shanghai (CN)

(21) Appl. No.: 18/117,068

(22) Filed: Mar. 3, 2023

(51) Int. Cl. G06T 7/00; G06T 7/10; G16H 30/40

(52) U.S. Cl. CPC G06T 7/0012; G06T 7/10; G16H 30/40; G06T 2207/30048

(57) ABSTRACT

Disclosed herein are systems, methods, and instrumentalities associated with cardiac motion tracking and/or analysis. In accordance with embodiments of the disclosure, the motion of a heart such as an anatomical component of the heart may be tracked through multiple medical images and a contour of the anatomical component may be outlined in the medical images and presented to a user. The user may adjust the contour in one or more of the medical images and the adjustment may trigger modifications of motion field(s) associated with the one or more medical images, re-tracking of the contour in the one or more medical images, and/or re-determination of a physiological characteristic (e.g., a myocardial strain) of the heart. The adjustment may be made selectively, for example, to a specific medical image or one or more additional medical images selected by the user, without triggering a modification of all of the medical images.

### BACKGROUND

Myocardial motion tracking and analysis can be used to detect early signs of cardiac dysfunction. The motion of the myocardium may be tracked by analyzing cardiac magnetic resonance (CMR) images (e.g., of a cardiac cine movie) of the myocardium captured over a time period and identifying the contour of the myocardium in those images, from which the movement or displacement of the myocardium across the time period may be determined. At times, the contour of the myocardium tracked using automatic means may need to be adjusted (e.g., to correct inaccuracies in the tracking), but conventional image analysis and motion tracking tools either do not allow a tracked contour to be adjusted at all or only allow the contour to be adjusted in a reference frame before propagating the adjustment to all other frames (e.g., without changing the underlying motion fields between the reference frame and the other frames). Such a correction technique is indirect and re-tracks the contour in every frame based on the reference frame even if some of those frames include no errors. Consequently, quality control in these conventional image analysis and motion tracking tools may be cumbersome, time-consuming and inaccurate, leading to waste of resources and even wrong strain analysis results.

### SUMMARY

Disclosed herein are systems, methods, and instrumentalities associated with cardiac motion tracking and/or analysis. In accordance with embodiments of the present disclosure, an apparatus configured to perform the motion tracking and/or analysis tasks may include a processor configured to present (e.g., via a monitor or a virtual reality headset) a first image of an anatomical structure and a second image of the anatomical structure, where the first image (e.g., a reference frame) may indicate a first tracked contour of the anatomical structure, the second image (e.g., a non-reference frame) may indicate a second tracked contour of the anatomical structure, and the second tracked contour may be determined based on the first tracked contour and a motion field between the first image and the second image. The processor may be further configured to receive an indication of a change to the second tracked contour, adjust the motion field between the first image and the second image in response to receiving the indication, and modify the second tracked contour of the anatomical structure in the second image based at least on the adjusted motion field. In examples, the first image may include a first segmentation mask for the anatomical structure and the first tracked contour of the anatomical structure may be indicated by the first segmentation mask (e.g., as a boundary of the first segmentation mask). Similarly, the second image may, in examples, include a second segmentation mask for the anatomical structure and the second tracked contour of the anatomical structure may be indicated by the second segmentation mask (e.g., as a boundary of the second segmentation mask). In examples, the indication of the change to the second tracked contour may be received via a user input such as a mouse click, a mouse movement, or a tactile input, and the change to the second tracked contour may include a movement of a part of the second tracked contour from a first location to a second location. In response to receiving the indication of change, the processor may adjust the motion field between the first image and the second image as well as the second tracked contour to reflect the movement of the part of the second tracked contour from the first location to the second location.

In examples, the processor being configured to adjust the motion field between the first image and the second image may comprise the processor being configured to identify a change to a feature point associated with the second tracked contour based on the movement of the part of the second tracked contour from the first location to the second location, determine a correction factor for the motion field between the first image and the second image, and adjust the motion field between the first image and the second image based on the correction factor.

In examples, the processor may be further configured to present a third image (e.g., another non-reference image) of the anatomical structure that may indicate a third tracked contour of the anatomical structure (e.g., the third image may include a third segmentation mask for the anatomical structure and the third tracked contour may be indicated as a boundary of the third segmentation mask). Similar to the second tracked contour, the third tracked contour may be determined based on the first tracked contour and a motion field between the first image and the third image, and, in response to receiving the indication of the change to the second tracked contour, the processor may modify the second tracked contour of the anatomical structure without modifying the third tracked contour of the anatomical structure. In examples, the first tracked contour may include a feature point that may also be included in the second tracked contour and the third tracked contour, and the processor may be further configured to determine that a change has occurred to the first tracked contour, determine a change to the feature point based on the change to the first tracked contour, and propagate the change to the feature point to at least one of the second image or the third image. The processor may, for example, receive an indication that the change to the feature point should be propagated to the third image and not to the second image. In response, the processor may determine a change to the feature point in the third image based on the change to the feature point in the first image and the motion field between the first image and the third image, and the processor may modify the third tracked contour of the anatomical structure based at least on the change to the feature point in the third image.

In examples, the anatomical structure described herein may include one or more parts of a heart, the first and second images may be CMR images of the heart, and the processor may be further configured to determine a strain value associated with the heart based on the tracked motion of the heart (e.g., based on the motion fields described herein).

### BRIEF DESCRIPTION OF THE DRAWINGS

A more detailed understanding of the examples disclosed herein may be obtained from the following description, given by way of example in conjunction with the accompanying drawing.

FIG. 1 is a simplified block diagram illustrating an example of tracking the contour of an anatomical structure in a series of medical images and modifying the tracked contour in one or more of the medical images based on an adjustment indication.

FIG. 2 is a flow diagram illustrating example operations that may be associated with tracking the contour of an anatomical structure in a series of medical images and modifying the tracked contour in one or more of the medical images based on an adjustment indication.

FIG. 3 is a flow diagram illustrating an example process for training an artificial neural network to perform the motion tracking and/or modification tasks described herein.

FIG. 4 is a simplified block diagram illustrating example components of an apparatus that may be configured to perform the motion tracking and/or modification tasks described herein.

### DETAILED DESCRIPTION

The present disclosure is illustrated by way of example, and not by way of limitation, in the figures of the accompanying drawings. A detailed description of illustrative embodiments will now be provided with reference to the various figures. Although this description provides detailed examples of possible implementations, it should be noted that the details are intended to be exemplary and in no way limit the scope of the application. The examples may be described in the context of CMR images, but those skilled in the art will understand that the techniques disclosed in those examples can also be applied to other types of images including. e.g., MR images of other anatomical structures, X-ray images, computed tomography (CT) images, photoacoustic tomography (PAT) images, etc.

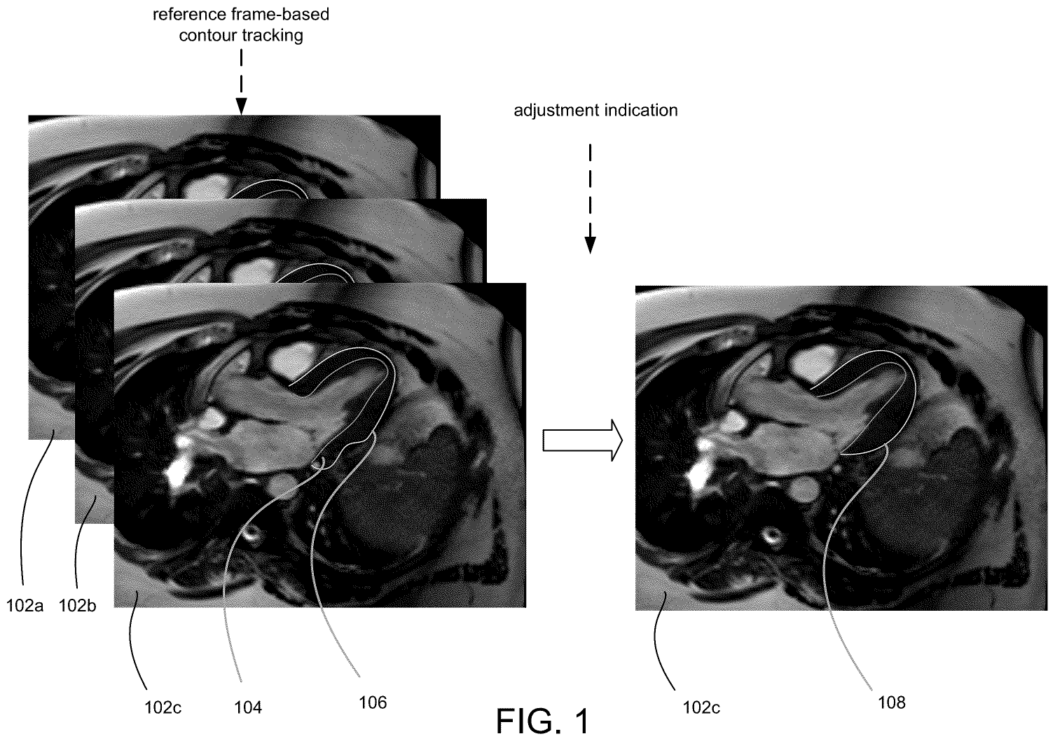

FIG. 1 illustrates an example of tracking the contour of an anatomical structure in a series of medical images and modifying the tracked contour in one or more of the medical images in response to receiving an adjustment indication. As shown, the series of medical images (e.g., 102a-c in the figure) may be CMR images depicting one or more anatomical structures of a heart such as a myocardium 104 of the heart. The CMR images may be captured over a time period t (e.g., as a cine movie), during which myocardium 104 may exhibit a certain motion. Such a motion may be automatically determined (e.g., from frame to frame) utilizing computer-based feature tracking techniques and the tracked motion may be used to determine physiological characteristics of the heart such as myocardial strains. In examples, the feature tracking may be performed using an artificial neural network (ANN) (e.g., a machine learning (ML) model) that may be trained for identifying intrinsic image features (e.g., anatomical boundaries and/or landmarks) associated with the myocardium and detecting changes to those features from one CRM frame to the next. During the tracking, a reference frame 102a (e.g., an end-diastolic phase frame) may be selected and the motion of the myocardium from reference frame 102a to a non-reference frame (e.g., 102b or 102c) may be tracked with reference to frame 102a. The motion may be indicated with a motion field (or a flow field), which may include values representing the displacements of a plurality of pixels between reference frame 102a and non-reference frame 102b/102c. The features identified by the ANN may also be used to determine a contour 106 of the myocardium in all or a subset of CMR images 102a-102c, and the contour may be outlined in those CMR images and presented to a user (e.g., via a display device such as a monitor or a virtual reality (VR) headset) for visualization of the motion tracking. For instance, the contour of the anatomical structure may be determined first on reference frame 102a (e.g., manually or automatically such as via a segmentation neural network) by identifying a plurality of feature points on the contour. The contour may then be tracked on another frame (e.g., 102b and/or 102c) by determining the respective movements of the same set of feature points on the other frame (e.g., the contours on the reference frame and the other frame may include a set of corresponding feature points) based on the motion field between the reference frame and the other frame.

In certain situations, the contour of the anatomical structure tracked using the techniques described above may need to be adjusted, e.g., to correct inaccuracies in the tracking. For instance, upon being presented with contour 106 of myocardium 104, a user may determine that one or more spots or segments of the contour in an image or frame may need to be adjusted to reflect the state (e.g., shape) of the myocardium more realistically. Such an image or frame may be a reference frame (e.g., frame 102a in FIG. 1) or a non-reference frame (e.g., frame 102c in FIG. 1), and the adjustment may be applicable to a specific frame (e.g., frame 102c) or to multiple frames (e.g., all or a subset of the images in the cine movie). The user may indicate the adjustment through a user interface that may be provided by the system or apparatus described herein. For example, the user may indicate the adjustment by clicking a computer mouse on a target spot or area, by dragging the computer mouse from an existing spot or area of the contour to the target spot or area of the contour, by providing a tactile input such as a finger tap or a finger movement over a touch screen, etc. The user may also select the frame(s) to which the adjustment may be applicable. For example, if the adjustment is indicated on a reference frame (e.g., 102a of FIG. 1), the user may additionally indicate which other frame or frames that the adjustment should be applied to (e.g., in addition to the reference frame). If the adjustment is indicated on a non-reference frame (e.g., 102c of FIG. 1), the adjustment may, by default, be applied only to that frame, although the user may also have the option of selecting other frame(s) for propagating the adjustment.

Based on the indication of adjustment, the contour of the anatomical structure may be modified (e.g., automatically) in the frame(s) selected by the user. For example, in response to receiving a user input or indication to change a part of the contour in non-reference frame 102c from a first location to a second location (e.g., via a mouse clicking or dragging), the motion field indicating the motion of the anatomical structure from reference frame 102a to non-reference frame 102c may be adjusted based on the indicated change and the contour of the anatomical structure may be re-tracked (e.g., modified from 106 to 108) based at least on the adjusted motion field. Such a technique may allow the contour to be modified in one or more individual frames, e.g., rather than re-tracking the contour in all of the frames based on the reference frame (e.g., the contour in frame 102c may be modified without modifying the contour in frame 102a or 102b). The technique may also allow for automatic adjustment of a motion field that may not be possible based on manual operations (e.g., human vision may not be able to discern motion field changes directly as it may do for edge or boundary changes). The accuracy of the motion tracking may thus be improved with the ability to adjust individual frames and/or motion fields directly rather than through the reference frame, and the resources used for the adjustment may also be reduced since some frames may not need to be changed. An adjustment may also be made to reference frame 102a (e.g., in addition to or instead of a non-reference frame), e.g., if the contour on the reference frame is determined by a user to be inaccurate. In such cases, the user may edit the contour on the reference frame and the editing may trigger a re-determination of the feature points associated with the edited contour on the reference frame. The user may additionally indicate (e.g., select) one or more non-reference frame(s) that may need to be re-tracked based on the reference frame such that the edit made on the reference frame may be propagated to those frames. The propagation may be accomplished, for example, by re-tracking corresponding feature points in the selected frames based on re-determined feature points on the reference frame and respective motion fields between the reference frame and the selected frames.

Using the motion tracking techniques described herein, continuity of the motion through time may be preserved and characteristics of the heart such as myocardial strains may be derived (e.g., adjusted from previously determined values) based on the modified contour(s). The strain values may be determined, for example, by tracking the motion of the myocardium throughout a cardiac cycle and calculating the myocardial strains (e.g., pixel-wise strain values) through a finite strain analysis of the myocardium (e.g., using one or more displacement gradient tensors calculated from the motion fields). In examples, respective aggregated strain values may be determined for multiple regions of interest (e.g., by calculating an average of the pixel-wise strain values in each region) and displayed/reported via a bullseye plot of the myocardium.

It should be noted that although the examples provided herein may be described with reference to a contour of the anatomical structure, those skilled in the art will appreciate that the disclosed techniques may also be used to process segmentation masks for the anatomical structure, in which case a contour of the anatomical structure may be determined by tracing the outside boundary of a corresponding segmentation mask. For instance, in the example shown in FIG. 1. images 102a-102c may include segmentation masks of the myocardium instead of or in addition to contours 106 of the myocardium, and the techniques described with respect to the figure may still be applicable since contours of the myocardium may be derived based on the outside boundary of the corresponding segmentation masks and the contours may be converted back into the segmentation masks by simply filling the inside of the contours. In the case where segmentation masks are included in images 102a-102c (e.g., instead of contours 106), the user may edit the segmentation masks, for example, by trimming certain parts of the segmentation masks or expanding the segmentation masks to include additional areas.

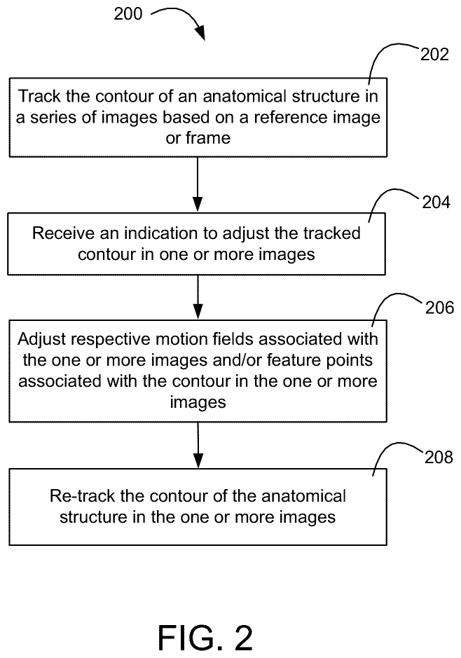

FIG. 2 illustrates example operations 200 that may be associated with tracking the contour of an anatomical structure in a series of medical images (e.g., a CRM cine movie) and modifying the tracked contour in one or more of the medical images in response to receiving an adjustment indication. As shown, the operations may include tracking the contour of the anatomical structure such as a myocardium at 202 based on a reference frame of the cine movie. Such tracking may be performed, for example, by identifying a set of feature points associated with a boundary of the anatomical structure in the reference frame and outlining the contour of the anatomical structure in the reference frame based on those feature points. The same set of feature points may then be identified in other frames of the cine movie and a respective motion field indicating the motion of the anatomical structure may be determined between the reference frame and each of the other frames based on the displacement of the feature points in those frames relative to the reference frame. The feature points may also be used to determine the contour of the anatomical structure in the other frames, e.g., in a similar manner as in the reference frame.

Operations 200 may also include receiving an indication to adjust the tracked contour of the anatomical structure in one or more images of the cine movie at 204. The indication may be received based on a user input that changes a part of the contour in a frame, e.g., from a first point to a second point, after the contour is presented to the user (e.g., via a display device). The user input may include, for example, a mouse click, a mouse movement, a tactile input, and/or the like that may change the shape, area, and/or orientation of the contour in the frame. And the user may also indicate which other frame or frames of the cine movie that the change should be propagated to. For example, upon adjusting the contour in the reference frame, the user may also select one or more other frames to propagate the adjustment to (e.g., in addition to the reference frame). As another example, the user may adjust the contour of the anatomical structure in a non-reference frame and choose to limit the adjustment only to that frame (e.g., without changing the contour in other frames).

Operation 200 may also include adjusting, at 206, the motion field associated with a frame on which the contour of the anatomical structure is to be re-tracked. For example, denoting the series of medical images described herein as I(t) (e.g., I(t_ref) may represent the reference frame) and the feature points (e.g., locations or coordinates of one or more boundary points of the anatomical structure) on the reference frame as P(t_ref), a flow field or motion field (e.g., a dense motion field) indicating the motion of the anatomical structure from the reference frame to a non-reference frame associated with time spot t may be determined (e.g., using a feature tracking technique as described herein) as F(t_ref, t)=G(I(t_ref), I(t)), where G( ) may represent a function (e.g., a mapping function realized through a pre-trained artificial neural network) for determining the motion field between the two frames. F may also be expressed as F(t_ref, t)=F(t_ref, t−1)⊕F(t−1, t), where t−1 may represent an intermediate time spot between t_ref and t, and ⊕ may represent a flow composite operator. As such, the following may be true: F(t_ref, t)*P(t_ref)˜=P(t) and F(t_ref. t)*I(t_ref)˜=I(t), where * may represent application of the motion field and ˜= may indicate that the contour or feature points tracked in the non-reference frame are substantially similar to those tracked in the reference frame.

If an adjustment is made to the contour in a non-reference frame at time t_i, it may indicate that feature points P(t_i) may not be accurate, which in turn may indicate that F(t_ref, t_i)*P(t_ref)!=Pₐ(t_i) and that the underlying motion field F(t_ref, t_i) may not be accurate, where Pₐ(t_i) may represent the accurate feature points corresponding to P(t_ref). Assuming that the user changes the feature points on the t_i frame to Pₐ(t_i), a motion estimate function K(P(t_i), Pₐ(t_i)) may be used to generate a motion field, F(t_i, t_i_a), between P(t_i) and Pₐ(t_i) such that F(t_i, t_i_a)*P(t_i)˜=Pₐ(t_i). F(t_i, t_i_a) may be considered as a correction factor to the original motion field F(t_ref. t_i) and may be used to change F(t_ref, t_i) to F_{c}(t_ref, t_i_a)=F(t_ref, t_i)⊕F(t_i, t_i_a), such that F_{c}(t_ref. t_i)*P(t_ref)˜=Pₐ(t_i). In examples, function K( ) may take two sets of feature points (or two sets of images or segmentation masks that may be generated based on the feature points) and derive the correction motion field Fe based on one or more interpolation and/or regularization techniques (e.g., based on sparse feature points). Functions G and K described herein may be realized using various techniques including, for example, artificial neural networks and/or image registration techniques. The two functions may be substantially similar (e.g., the same), e.g., with respect to estimating a motion between two images, two masks, or two sets of points. For example, function G may be designed to take two images as inputs and output a motion field, while function K may be designed to take two segmentation masks or two sets of feature points as inputs and output a motion field. In cases where G and K are realized via artificial neural networks, the two networks may be trained in substantially similar manners.

If an adjustment is made to the contour in the reference frame, feature points associated with the contour in the reference frame may be re-determined and the feature points may be re-tracked in one or more other frames (e.g., the contours in those frames may be modified), for example, based on previously determined motion fields between the reference frame and the r-tracked frames (e.g., without changing the motion fields). The user may select the frame(s) to be re-tracked. The user may also select the frame(s) in which an original contour is to be kept, in which case the feature points associated with the original contour may be treated as Pₐ(t) described herein and the motion fields between the reference frame and the unchanged frames may be updated to reflect the difference between the feature points in the reference frame and the feature points in the unchanged frames.

At 208, the contour of the anatomical structure may be re-tracked in one or more frames. The re-tracking may be performed in one or more frames selected by a user (e.g., if the contour in the reference frame is adjusted) or in a specific frame (e.g., if the contour is a non-reference frame is adjusted). In the former case, feature points associated with the contour may be re-tracked in each selected frame based on the reference frame (e.g., without changing the motion fields associated with those frames), while in the latter case the motion field between the specific frame and the reference frame may be corrected to reflect the change made by the user.

The feature tracking and/or motion field adjustment operations described herein may be performed using an artificial neural network such as a convolutional neural network (CNN). Such a CNN may include an input layer, one or more convolutional layers, one or more pooling layers, and/or one or more fully-connected layers. The input layer may be configured to receive an input image while each of the convolutional layers may include a plurality of convolution kernels or filters with respective weights for extracting features associated with an anatomical structure from the input image. The convolutional layers may be followed by batch normalization and/or linear or non-linear activation (e.g., such as a rectified linear unit (ReLU) activation), and the features extracted through the convolution operations may be down-sampled through one or more pooling layers to obtain a representation of the features, for example, in the form of a feature vector or a feature map. In examples, the CNN may further include one or more un-pooling layers and one or more transposed convolutional layers. Through the un-pooling layers, the features extracted through the operations described above may be up-sampled and the up-sampled features may be further processed through the one or more transposed convolutional layers (e.g., via a plurality of deconvolution operations) to derive an up-scaled or dense feature map or feature vector, which may then be used to predict a contour of the anatomical structure or a motion field indicating a motion of the anatomical structure between two images.

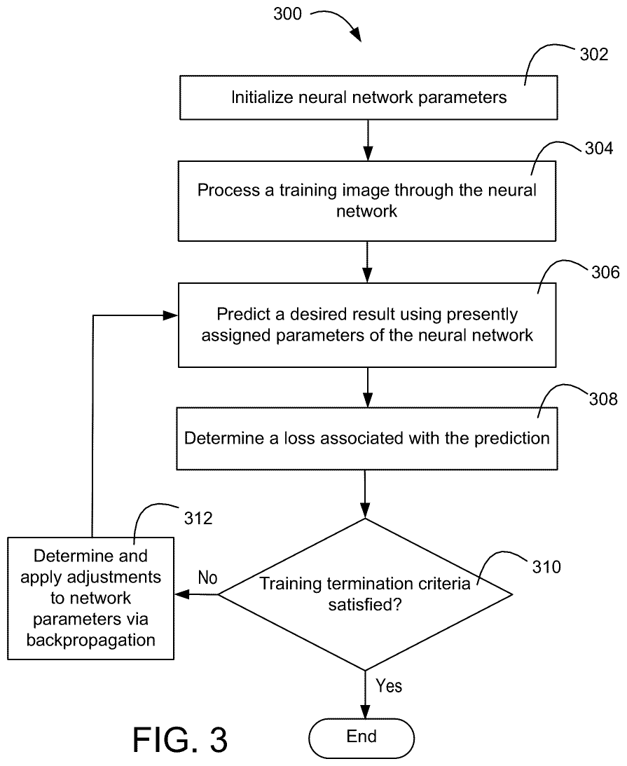

FIG. 3 illustrates an example process 300 for training an artificial neural network (e.g., the CNN described above) to perform one or more of the tasks described herein. As shown, the training process may include initializing parameters of the neural network (e.g., weights associated with various layers of the neural network) at 302, for example, based on samples from one or more probability distributions or parameter values of another neural network having a similar architecture. The training process may further include processing an input training image (e.g., a CMR image depicting a myocardium) at 304 using presently assigned parameters of the neural network and making a prediction for a desired result (e.g., a set of feature points, a contour of the myocardium, a motion field, etc.) at 306. The predicted result may be compared to a corresponding ground truth at 308 to determine a loss associated with the prediction. Such a loss may be determined, for example, based on mean squared errors between the predicted result and the ground truth. At 310, the loss may be evaluated to determine whether one or more training termination criteria are satisfied. For example, the training termination criteria may be determined to be satisfied if the loss is below a threshold value or if the change in the loss between two training iterations falls below a threshold value. If the determination at 310 is that the termination criteria are satisfied, the training may end; otherwise, the presently assigned network parameters may be adjusted at 312, for example, by backpropagating a gradient descent of the loss through the network before the training returns to 306.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

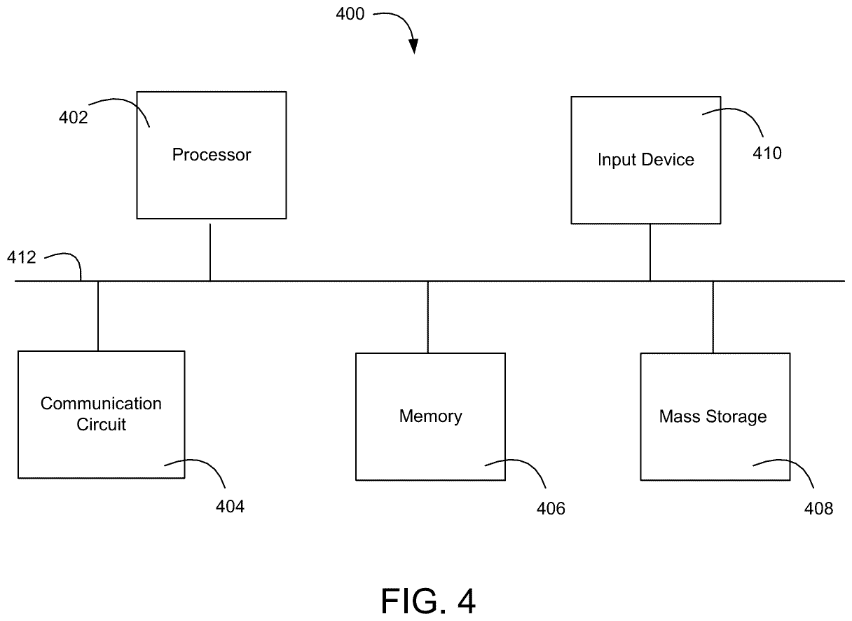

The systems, methods, and/or instrumentalities described herein may be implemented using one or more processors, one or more storage devices, and/or other suitable accessory devices such as display devices, communication devices, input/output devices, etc. FIG. 4 is a block diagram illustrating an example apparatus 400 that may be configured to perform the tasks described herein. As shown, apparatus 400 may include a processor (e.g., one or more processors) 402, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 400 may further include a communication circuit 404, a memory 406, a mass storage device 408, an input device 410, and/or a communication link 412 (e.g., a communication bus) over which the one or more components shown in the figure may exchange information.

Communication circuit 404 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 406 may include a storage medium (e.g., a non-transitory storage medium) configured to store machine-readable instructions that, when executed, cause processor 402 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 408 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 402. Input device 410 may include a keyboard, a mouse, a voice-controlled input device, a touch sensitive input device (e.g., a touch screen), and/or the like for receiving user inputs to apparatus 400.

It should be noted that apparatus 400 may operate as a standalone device or may be connected (e.g., networked, or clustered) with other computation devices to perform the functions described herein. And even though only one instance of each component is shown in FIG. 4, a skilled person in the art will understand that apparatus 400 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
a processor configured to:
present a first image of an anatomical structure and a second image of the anatomical structure, wherein the first image indicates a first tracked contour of the anatomical structure, the second image indicates a second tracked contour of the anatomical structure, and the second tracked contour is determined based on the first tracked contour and a motion field between the first image and the second image;
receive an indication of a change to the second tracked contour;
adjust the motion field between the first image and the second image in response to receiving the indication of the change to the second tracked contour; and
modify the second tracked contour of the anatomical structure in the second image based at least on the adjusted motion field.

2. The apparatus of claim 1, wherein the first image includes a first segmentation mask for the anatomical structure that indicates the first tracked contour of the anatomical structure, and wherein the second image includes a second segmentation mask for the anatomical structure that indicates the second tracked contour of the anatomical structure.

3. The apparatus of claim 1, wherein the change to the second tracked contour includes a movement of a part of the second tracked contour from a first location to a second location and wherein the processor is configured to adjust the motion field based at least on the movement of the part of the second tracked contour from the first location to the second location.

4. The apparatus of claim 3, wherein the indication of the change to the second tracked contour is received based on a user input that includes at least one of a mouse click, a mouse movement, or a tactile input.

5. The apparatus of claim 3, wherein the processor being configured to adjust the motion field based at least on the movement of the part of the second tracked contour from the first location to the second location comprises the processor being configured to:
identify a change to a feature point associated with the second tracked contour based on the movement of the part of the second tracked contour;
determine a correction factor for the motion field between the first image and the second image; and
adjust the motion field between the first image and the second image based on the correction factor.

6. The apparatus of claim 1, wherein the processor is further configured to present a third image of the anatomical structure that includes a third tracked contour of the anatomical structure determined based on the first tracked contour and a motion field between the first image and the third image, and wherein, in response to receiving the indication of the change to the second tracked contour, the processor is configured to modify the second tracked contour of the anatomical structure without modifying the third tracked contour of the anatomical structure.

7. The apparatus of claim 6, wherein the first tracked contour includes a feature point that is also included in the second tracked contour and the third tracked contour, and wherein the processor is further configured to:
determine that a change has occurred to the first tracked contour;
determine a change to the feature point based on the change to the first tracked contour; and
propagate the change to the feature point to at least one of the second image or the third image.

8. The apparatus of claim 7, wherein the processor being configured to propagate the change to the feature point to at least one of the second image or the third image comprises the processor being configured to receive an indication that the change to the feature point is to be propagated to the third image and not to the second image.

9. The apparatus of claim 8, wherein the processor being configured to propagate the change to the feature point to at least one of the second image or the third image further comprises the processor being configured to:
determine a change to the feature point in the third image based on the change to the feature point in the first image and the motion field between the first image and the third image; and
modify the third tracked contour of the anatomical structure based at least on the change to the feature point in the third image.

10. The apparatus of claim 1, wherein the anatomical structure includes one or more parts of a heart and the processor is further configured to determine a strain value associated with the heart based at least on the adjusted motion field.

11. A method of processing medical images, the method comprising:
presenting a first image of an anatomical structure and a second image of the anatomical structure, wherein the first image indicates a first tracked contour of the anatomical structure, the second image indicates a second tracked contour of the anatomical structure, and the second tracked contour is determined based on the first tracked contour and a motion field between the first image and the second image;
receiving an indication of a change to the second tracked contour;
adjusting the motion field between the first image and the second image in response to receiving the indication of the change to the second tracked contour; and
modifying the second tracked contour of the anatomical structure in the second image based at least on the adjusted motion field.

12. The method of claim 11, wherein the first image includes a first segmentation mask for the anatomical structure that indicates the first tracked contour of the anatomical structure, and wherein the second image includes a second segmentation mask for the anatomical structure that indicates the second tracked contour of the anatomical structure.

13. The method of claim 11, wherein the change to the second tracked contour includes a movement of a part of the second tracked contour from a first location to a second location and wherein the motion field is adjusted based at least on the movement of the part of the second tracked contour from the first location to the second location.

14. The method of claim 13, wherein the indication of the change to the second tracked contour is received based on a user input that includes at least one of a mouse click, a mouse movement, or a tactile input.

15. The method of claim 13, wherein adjusting the motion field based at least on the movement of the part of the second tracked contour from the first location to the second location comprises:
identifying a change to a feature point associated with the second tracked contour based on the movement of the part of the second tracked contour;
determining a correction factor for the motion field between the first image and the second image; and
adjusting the motion field between the first image and the second image based on the correction factor.

16. The method of claim 11, further comprising presenting a third image of the anatomical structure that indicates a third tracked contour of the anatomical structure determined based on the first tracked contour and a motion field between the first image and the third image, and wherein, in response to receiving the indication of the change to the second tracked contour, the second tracked contour of the anatomical structure is modified without modifying the third tracked contour of the anatomical structure.

17. The method of claim 16, wherein the first tracked contour includes a feature point that is also included in the second tracked contour and the third tracked contour, and wherein the method further comprises:
determining that a change has occurred to the first tracked contour;
determining a change to the feature point based on the change to the first tracked contour; and
propagating the change to the feature point to at least one of the second image or the third image.

18. The method of claim 17, wherein propagating the change to the feature point to at least one of the second image or the third image comprises:
receiving an indication that the change to the feature point is to be propagated to the third image and not to the second image;
determining a change to the feature point in the third image based on the change to the feature point in the first image and the motion field between the first image and the third image; and
modifying the third tracked contour of the anatomical structure based at least on the change to the feature point in the third image.

19. The method of claim 11, wherein the anatomical structure includes one or more parts of a heart and the method further includes determining a strain value associated with the heart based at least on the adjusted motion field.

20. A non-transitory computer-readable medium comprising instructions that, when executed by a processor included in a computing device, cause the processor to implement the method of claim 11.
